(19) United States
(12) Patent Application Publication
(10) Pub. No.: US 2012/0220654 A1
(43) Pub. Date: Aug. 30, 2012

(54) THERAPEUTIC SOLUTION CONCENTRATE

(76) Inventors: Joseph P. Reo, Lakeland, TN (US); Kevin C. Fowler, Millington, TN (US); Mohammed A. Kabir, Cary, NC (US); Michael P. Tune, Arlington, TN (US); Dennis Nelson, Memphis, TN (US); Anna W. Tai, Germantown, TN (US); Nanhye Kim, Germantown, TN (US)

(21) Appl. No.: 13/279,801

(22) Filed: Oct. 24, 2011

Related U.S. Application Data
(60) Provisional application No. 61/407,969, filed on Oct. 29, 2010.

(51) Int. Cl. A61K 31/235; A61P 1/10

(52) U.S. Cl. 514/544

(57) ABSTRACT

A concentrated poly(ethylene glycol) solution that has good stability for extended storage is provided for use as a laxative. The concentrated solution comprises water, sodium benzoate, poly(ethylene glycol), and enough acid to yield a solution having a pH at 25° C. in the range 3.0-5.0. It is prepared and packaged to minimize the accumulation of degradation products.

### BACKGROUND OF THE INVENTION

Polyethylene glycol (PEG) based laxatives are widely used to treat constipation. PEG having an average molecular weight of 3350 (PEG 3350) is normally used in the formulations that are marketed as laxatives. PEG 3350 based laxatives are osmotic laxatives, which cause a sufficient amount of water to be absorbed from the body into the bowel, so that a bowel movement occurs within a period of hours. These are preferred over stimulant laxatives, which work by acting through the nervous system to stimulate the large intestine, causing cramping and discomfort.

The recommended dose of PEG 3350 in a laxative formulation is 17 g of PEG 3350 in 4-8 ounces of solution. The commercial products are sold as powders which are dissolved in water or other beverage to make a solution shortly before they are used. Powders are inconvenient because they do not dissolve instantaneously. The patient must first mix the powder with water or other beverage and then wait for it to dissolve. Stirring one or more times is normally required to completely dissolve the solid ingredients. The preparation takes only a few minutes (generally less than 5 minutes), but this is enough of an inconvenience that a prospective patient may choose another laxative to avoid expending the extra time and effort.

Pre-mixed PEG solutions would be far more convenient to use than powders because they eliminate the step of dissolving 17 g of the PEG 3350 powder in a beverage. Pre-mixed PEG solutions for oral administration are not currently available, possibly because of concerns about stability. Also, pre-mixed solutions in which the dose is 17 g of PEG 3350 in 8 ounces of liquid are inconvenient in the sense that a container with several 8 ounce doses would be bulky. An alternative approach is to have a highly concentrated pre-mixed solution of PEG 3350 on hand. The concentrated solution can then be taken directly or diluted as needed. A concentrated liquid PEG solution would mix rapidly with any beverage to make a dilute solution having the appropriate concentration. The concentrated solution would have less volume and less weight than a dilute solution having the same number of doses.

A recently published patent application (WO 2005/049049, Aaronson et al.) suggests that concentrated PEG solutions may be more stable than dilute solutions. The concentrated solution of Aaronson et al. was described as “shelf stable,” being suitable for storage for at least six months at room temperature. The solution of Aaronson et al. was reported to be “a clear, colorless, generally tasteless and odorless liquid.” However, the Aaronson et al. publication did not recognize or mention that there are degradation products that can form even in the concentrated solution.

The applicants of the instant application have discovered that degradation products can form in the concentrated PEG solutions and that the degradation products can accumulate over time to levels that are potentially harmful to patients who consume the solutions. The solution concentrate that is disclosed herein has been formulated to control and prevent the accumulation of degradation products over an extended time so that the patient can use the solution concentrate with confidence that the product is safe even after it has been stored for an extended time before it is used (e.g. one to two years).

### SUMMARY OF THE INVENTION

A solution concentrate is provided herein that has good stability for extended storage. It can be taken directly or diluted to a lower concentration (for example by dilution of 1 fluid ounce of solution with water or other beverage to about 8 fluid ounces) and then can be used in diluted form as an oral laxative solution. The solution concentrate comprises (w/w) (a) 37%-57% water; (b) 0.05%-0.36% sodium benzoate; (c) 41%-62% polyethylene glycol having an average molecular weight of 2680-4020 Daltons; and (d) a pharmaceutically acceptable acid in an amount sufficient to yield a solution having a pH at 25° C. in the range 3.0-5.0.

The solution concentrate is packaged in a container that is sealed under conditions such that the oxygen content of the packaged solution concentrate is no more than 5 ppm, and preferably no more than 3 ppm, at the time when the container is first sealed. Furthermore, the container is constructed so that it limits oxygen uptake from the atmosphere so that the amount of oxygen in the container does not exceed 5 ppm, and preferably does not exceed 3 ppm, during the time the container remains sealed, which may be for a period of time up to two years.

### DETAILED DESCRIPTION OF THE INVENTION

In many embodiments, the container is a 1-50 ounce bottle that allows an uptake of oxygen from the air of no more than about 0.1 cc/bottle/day at room temperature and one atmosphere of pressure. Ambient air has an oxygen concentration of approximately 21%. A desirable bottle size is 1-30 ounces. Preferred bottle sizes are sizes that are readily available and made of a material that resists permeation by oxygen, such as poly(ethylene terephthalate) (PET), such as 1-1.5 ounces, which would contain a single dose, 12 and 26 ounces, and 7 ounces, which is a size of bottle that is used in the EU. The bottle is sealed under conditions such that the oxygen content of the packaged solution concentrate is no more than 5 ppm, and preferably less than 3 ppm, at the time the container is initially sealed. The amount of oxygen in the container remains at a level of no more than 5 ppm, and preferably no more than 3 ppm, from the time when the container is first sealed until the sealed container is opened at a time up to two years later.

The rate at which oxygen permeates into the bottle depends on the material that is used in the construction of the bottle. A polymer having an oxygen transmission rate less than about 100 cc-mil/100 in²-day when measured in the form of a sheet or film is generally suitable for use in the bottles containing the therapeutic solution concentrate. The rate of permeation also depends on the thickness of the walls of the bottle. A preferred material of construction of the bottle is poly(ethylene terephthalate) (PET), which has very good properties with respect to penetration by oxygen and is also readily available in the form of bottles. Other polymer resins and glass can also be used provided they are sufficiently resistant to penetration by oxygen and are otherwise suitable for use as a container for a medication. High density polyethylene (HDPE) can also be used, but HDPE is much more permeable to oxygen, so that the walls of the container would have to be much thicker to limit oxygen penetration and resulting PEG degradation. Alternatively, the walls of an HDPE container can be coated with a material that resists permeation by oxygen, such as a thin metal film or a thin coating of a polymer film that resists permeation by oxygen. HDPE and other polymers can also be made into bottles that are permeation resistant to oxygen by laminating layers of polymers that are resistant to oxygen permeation, such as ethylene vinyl alcohol laminated between two layers of HDPE. HDPE and other polymers can also be made more resistant to penetration by oxygen by blending the resins with certain metal oxides and clays, such as montmorillonite and mica.

The solution concentrate may contain one or more of the following degradation products in a detectable amount, where the detectable degradation product may be present in an amount no greater than the following amounts: (w/w): formic acid, 0.5%, preferably no more than 0.3%; ethylene glycol, 0.10%, preferably no more than 365 ppm, preferably no more than 180 ppm; diethylene glycol, 0.10%, preferably no more than 590 ppm, preferably no more than 180 ppm; formaldehyde, 200 ppm, preferably no more than 175 ppm, preferably no more than 70 ppm; and acetaldehyde, 200 ppm, preferably no more than 115 ppm, preferably no more than 70 ppm.

The amount of the active ingredient PEG 3350 remains at a level between 90 and 110% of the amount originally charged (47%-57% of the solution). In preferred embodiments the PEG 3350 remains at a level between 93%-107% of the amount that was charged (48%-55% of the solution.

In general, it is very difficult to completely prevent degradation of the PEG 3350. As a result, one or more degradation products will generally be present in a detectable amount in the solution concentrate. These degradation products are believed to be degradation products of PEG 3350. The amounts of these degradation products increase with time, with the amounts depending on the temperature, the duration of time, and the kind and size of the container. The amounts of the degradation products remain below the limits specified above when the solution is packaged as described above, under conditions where the level of oxygen is low when the container is sealed, and where the container is designed to minimize permeation of oxygen from the air into the container.

In many embodiments of the invention as described above, the solution concentrate comprises (w/w) (a) 42%-52% water; (b) 0.27-0.33% sodium benzoate; and (c) 46%-57% polyethylene glycol having an average molecular weight of 3015-3685 Daltons.

In preferred embodiments, the solution concentrate comprises about (w/w) (a) 47% water; (b) 0.3% sodium benzoate; and (c) 52% polyethylene glycol having an average molecular weight of 3350 Daltons.

In the embodiments described above, the solution concentrate contains enough acid that the pH is in the range 3.0-5.0. In preferred embodiments, the pH is in the range 3.5-5.0.

In some embodiments of the solution concentrate, the pharmaceutically acceptable acid is a mineral acid selected from sulfuric acid, phosphoric acid, and hydrochloric acid.

In preferred embodiments of the solution concentrate, the pharmaceutically acceptable acid is hydrochloric acid.

In some embodiments of the solution concentrate, the solution concentrate contains only a mineral acid and does not contain an organic acid. In some preferred embodiments of the solution concentrate, the solution concentrate contains hydrochloric acid as the only acid component.

In some embodiments of the solution concentrate, the solution concentrate may contain one or more pharmaceutically acceptable chelating agents, for example citric acid, malic acid or disodium edetate (EDTA). In certain preferred embodiments the chelating agent is present in an amount between 0.01% to 2.0% w/w. In other preferred embodiments the chelating agent is present in an amount between 0.01% to 0.1% w/w. In additional preferred embodiments chelating agent is present in an amount of 0.01% w/w, 0.02% w/w, 0.025% w/w, 0.05% w/w, 0.075% w/w, or 0.1% w/w.

In a preferred embodiment, the solution concentrate when it is first manufactured comprises (w/w) (a) 47.7% water; (b) 0.30% sodium benzoate; (c) 51.7% polyethylene glycol having an average molecular weight of 3350 Daltons, and (d) enough hydrochloric acid to provide a solution with a pH of 4.0 at 25° C.

Defininitions, Abbreviations, Acronyms

AcH is acetaldehyde.

DEG is diethylene glycol.

EG is ethylene glycol.

EPA is the Environmental Protection Agency

GRAS is the abbreviation for Generally Recognized As Safe.

ND means not determined

NF means National Formulary

NQ means not quantifiable.

PEG is an abbreviaton for polyethylene glycol.

PEG 3350 refers to PEG 3350 NF, which is the grade of polyethylene glycol having an average molecular weight of 3350 Daltons that is of suitable quality and purity that it can be used in a laxative solution. PEG 3350 is available in powdered form for use by consumers as a laxative immediately after it is dissolved, but up to now it has not been available to consumers in solution form.

RfD means Reference Dose.

RH is relative humidity.

TDI is the Tolerable Daily Intake, which is set by Health Canada.

USP means the United States Pharmacopeia.

w/w when used with % means weight %.

Color and Appearance. The solution concentrate is colorless and clear to slightly hazy when formulated as described above. When formulated with other preservatives such as potassium sorbate or in the presence of antioxidants, such as sodium metabisulfite, vitamin E, malic acid, propyl gallate, or sodium thiosulfate, the solution has at least some amount of color. The color is usually yellow, and may increase over time, with the solution becoming darker.

Utility. The solution concentrate described herein is an alternative dosage form that may be used instead of powdered PEG 3350 as a laxative. It is expected to provide the same relief that is obtained using solutions of the PEG 3350 in powdered form. Either formulation (liquid or powder) is taken orally as a solution once a day or as directed by a physician to relieve occasional constipation (irregularity).

A single dose of either formulation comprises 17 g of PEG 3350 as either a measured amount of powder or as a component in 30 mL of the solution concentrate. If the powder is used, it is mixed with 4-8 ounces of beverage. When the solution concentrate is used, 30 mL of the solution concentrate (about one fluid ounce) can be taken directly or mixed with 4-8 ounces of beverage, which the patient then drinks. In either case, the dose of PEG 3350 that is consumed is usually about 17 g.

The solution concentrate is more convenient for the patient, since the patient can take it directly without dilution or mix the appropriate volume of the solution concentrate with 4-8 ounces of beverage, which is then consumed without the need for stirring. A small measuring vessel can be provided with the solution concentrate to further simplify the process.

### Control of Degradation Products

PEG 3350 is poly(ethylene glycol), which is also known as poly(oxyethylene), having an average molecular weight of 3350 Daltons. PEG 3350 NF is the grade of PEG that is used in the solution concentrate as it is of suitable quality and purity that it can be used in an oral laxative solution. The molecular formula is represented as follows:

H(OCH₂CH₂)ₙOH,

where “n” represents the number of monomer units in the polymer chain.

The applicants have found that several degradation products accumulate in PEG 3350 solution concentrate over an extended period of storage, including acetaldehyde, formaldehyde, ethylene glycol, diethylene glycol, and formic acid. These degradation products can be harmful or toxic when consumed in large quantities or in high concentrations. Limits on the amounts of these chemicals in the solution concentrate are recommended, as summarized below.

Acetaldehyde and the other degradation products appear to accumulate from at least two sources in the solution concentrate. First, it has been discovered that acetaldehyde appears in the solution concentrate as soon as the solution concentrate is prepared. The other degradation products may also be present in the freshly prepared solution. A possible explanation is that acetaldehyde is released from the PEG 3350 when it is dissolved through a chemical reaction of an impurity in the PEG 3350. Alternatively, the acetaldehyde may have been adsorbed or entrapped in the solid PEG 3350 and was released when the PEG 3350 was dissolved. In several examples in which the amount of acetaldehyde was measured before the start of stability tests, the amount of acetaldehyde was found to be in the range of 79-102 ppm in freshly prepared batches of solution concentrate. The other degradation products (formaldehyde, ethylene glycol, and diethylene glycol) are also sometimes present, but in smaller amounts than acetaldehyde. Although the mechanism that results in the presence of acetaldehyde and the other degradation products in freshly made solutions is not known, it has been observed that the amount of acetaldehyde that is formed when the solution concentrate is mixed varies depending on the source of PEG 3350. A source of PEG 3350 that yields less than 60 ppm of acetaldehyde when the solution concentrate is initially mixed has been identified (see Example 6). A preferred supplier of PEG 3350 may need to meet a product standard specifying that the concentrated solution that is prepared using the PEG 3350 contains no more than 60 ppm acetaldehyde, 60 ppm formaldehyde, 100 ppm ethylene glycol, and 100 ppm diethylene glycol when it is first prepared.

Degradation Products

Acetaldehyde appears to be a product resulting from degradation of PEG 3350 and also appears to be an impurity that is present in the PEG 3350 or is released when the solid PEG 3350 is dissolved to form the solution concentrate. Other degradation products that are formed were mentioned previously, including formaldehyde, formic acid, ethylene glycol, and diethylene glycol. The PEG 3350 in the solution concentrate can undergo slow degradation over an extended period of time. Over a period of 1-2 years, degradation can lead to decreased potency of the PEG 3350 as a laxative and also to the accumulation of degradation products which are potentially harmful.

The primary degradation pathway of PEG in the solution concentrate is believed to be an oxidative decomposition. The oxidation of PEG is believed to proceed by a free radical chain mechanism comprising steps of initiation, propagation, branching, rearrangement, and termination. The free radical chain reaction may be propagated by oxygen, which can form new peroxide radicals by coupling with free radicals that are already in solution. The new peroxide radicals then can react with the polymer chains to form hydroperoxides and more radicals. To the extent that the chain reaction relies on oxygen molecules to continue propagation of the chain reaction, reduction or removal of oxygen may significantly reduce the rate of degradation of PEG 3350. The extent to which degradation occurs is also determined by other factors such as temperature, the initiator, the ratio of initiator to substrate, the solvent, and the concentration of PEG. Because PEG is a polymer, reactions can occur at the same time at more than one site on the polymer molecule, leading to multiple propagation pathways and multiple by-products, which are not readily predicted.

Control of microbial growth is also important for minimizing degradation reactions. It was reported in the Aaronson et al. patent publication that concentrated PEG solutions are chemically stable and do not support microbial growth. Further, one skilled in the art would not expect that a preservative is necessary in a solution with the osmolality value of the solution concentrate (about 4400 mOsmoles/kg). However, the applicants have discovered that the concentrated PEG 3350 solutions do slowly degrade in the absence of a preservative. Sodium benzoate is still needed as a preservative to inhibit the growth of all forms of microbes. When solutions were formulated without sodium benzoate, acceptance criteria specified in the European Pharmacopeia (EP) were not met with regards to mold growth. There was a less than 1.0 log reduction from the initial count of Aspergillus brasiliensis at Day 14, and there was an increase in mold between Day 14 and Day 28. In contrast, the applicants have found that solutions formulated with 0.24% and 0.30% sodium benzoate met all United States

Pharmacopeia (USP) and EP acceptance criteria for preservative effectiveness. The pH needs to be below 5.0 for the sodium benzoate to inhibit growth of bacteria, molds, and yeasts, according to the USP and the EP standards. The pH of the solution concentrate is kept at 3.0-5.0, and preferably 3.5-5.0, to ensure that the growth of bacteria, molds, and yeasts is fully inhibited. A preferred pH of freshly manufactured solution concentrate is 4.0.

### Recommended Limits of Degradation Products

Ethylene Glycol (EG). Ethylene Glycol is a Class 2 solvent and needs to be limited to protect the consumer from adverse effects. Based on ICH “Guidance for Industry—Q3C Impurities: Residual Solvents,” for products administered at a dose greater than 10 g/day, a preferred (conservative) limit of 365 ppm was set for EG. In certain embodiments of the invention a further preferred limit for EG is180 ppm.

Diethylene Glycol (DEG). The USP NF monograph for PEG having a nominal molecular weight not more than 1000 suggests a limit of impurities of no more than 0.25% for EG and DEG combined. As no limits were stated for DEG in the guidance document, a limit was set at 0.1% for DEG, and a preferred limit was set at 590 ppm. In certain embodiments of the invention a further preferred limit for DEG is180 ppm.

Formic Acid. Formic acid is listed as a Class 3 solvent within guidelines by ICH, “Guidance for Industry—Q3C Impurities: Residual Solvents,” December 1997. Class 3 residual solvents are considered acceptable without justification at a level up to 5000 ppm. Using the equation outlined in the guidance document, a preferred limit of 0.3% was set for formic acid based on the dose of 17 grams.

Acetaldehyde. Acetaldehyde is a naturally occurring substance which has been designated as Generally Recognized As Safe (GRAS) for use as a flavoring ingredient in certain foods. However, acetaldehyde is also listed as a possible carcinogen to humans through inhalation, though not through ingestion. Acetaldehyde can give rise to other degradation products, including formic acid and formaldehyde, and possibly condensation products. Based on the Tolerable Daily Intake (TDI) established by the Scientific Committee on Food, a conservative limit of human ingestion of acetaldehyde was set at 6 mg/day for this product. This corresponds to a concentration of 353 ppm acetaldehyde as a safe limit in the solution concentrate. A preferred (more conservative) limit for acetaldehyde was set at 115 ppm to allow for the possibility that the solution concentrate might be administered to smaller (20 kg) pediatric patients. In certain embodiments of the invention a further preferred limit for acetaldehyde is 70 ppm.

Formaldehyde. Formaldehyde is recognized by the International Agency for Research on Cancer (IARC) as carcinogenic to humans by inhalation, but the weight of evidence indicates that formaldehyde is not carcinogenic by the oral route. Based on the TDI set by

Health Canada and the RfD set by EPA, a conservative limit was set on the formaldehyde level in the product of 9 mg/day. This corresponds to a concentration of 529 ppm formaldehyde as a safe level in the solution concentrate. A more conservative limit of 175 ppm allows for the possibility of administration to children. In certain embodiments of the invention a further preferred limit for formaldehyde is 70 ppm.

### Control of Oxidation Reactions

Exclusion of oxygen is important to ensure that the amount of by-product resulting from degradation caused by oxidation or catalyzed by oxygen is maintained at a low level. The accumulation of degradation products, including by-products of oxidation, must be kept at a low enough level that the degradation products remain at levels that are safe over an extended period of time in storage, equivalent to two years at room temperature (25° C. and 40% RH) or 6 months at 40° C. and 20% RH. The latter range is the accelerated test for 2 years at room temperature as per ICH Guideline Q1A (International Conference on Harmonization of Technical Requirements for Registration of Pharmaceuticals for Human Use). This is achieved by one or more of the following strategies: (1) minimizing the volume of the headspace when the bottle is sealed so that the amount of any air that remains in the headspace after sealing is too small to cause significant degradation; (2) storage of the solution concentrate in a container made of a material that acts as a barrier to penetration by oxygen, such as poly(ethylene terephthalate) (PET), so that permeation of oxygen into the bottle is limited; (3) use of antioxidants; and (4) exclusion of oxygen when the product is bottled by filling the headspace or purging the bottle with an inert gas such as nitrogen.

The use of antioxidants, such as sodium metabisulfite, vitamin E, malic acid, propyl gallate, and sodium thiosulfate, generally causes some coloration (e.g. yellowing) of the solution. Filling the headspace with nitrogen or purging the bottle with nitrogen is a less preferred method of reducing the oxygen content of the bottled solutions of solution concentrate because it is difficult to use in large scale processes. The headspace volume can be minimized by filling the bottle almost to the top with liquid, leaving enough space above the liquid so that the bottle can be capped and sealed, so that the residual air in the bottle is at an acceptable level after sealing.

### Effect of Bottle Composition and Size on Stability of the Solution Concentrate.

Studies were carried out under controlled conditions to determine the stabilities of the solution concentrate during periods of storage. It was discovered that the bottle compositions and the bottle sizes have a measurable effect on the stability of the solution concentrate during storage. Developmental stability studies were performed on samples of the solution concentrate in 1, 12.1, and 26.3 ounce high density polyethylene (HDPE) bottles. The 1-ounce bottles exhibited larger amounts of degradation than did the larger sized bottles. This is consistent with the hypothesis that the degradation reactions are mediated by air oxidation. Smaller bottles have a larger ratio of surface area to volume than do the larger 12.1 and 26.3 ounce bottles, and therefore would be subject to permeation of larger amounts of oxygen per unit volume of solution.

The variations in surface area to volume are shown in the table below for the three sizes of bottles of PET and HDPE that were used in the studies. The 1 ounce HDPE bottles have about 3 times as large a surface area to volume ratio as the 26.3 ounce HDPE bottles. The corresponding ratio of 1.5 ounce PET bottles compared with 26.3 ounce PET bottles is about 2.5.

| | Surface Area | Volume | Surface Area: |
|---|---|---|---|
| Bottle Size and Material | (sq. in.) | (cc) | Volume |
| 1.5 oz PET | 11.54 | 48.5 | 0.237 |
| 12 oz PET | 51.256 | 420.9 | 0.121 |
| 26 oz PET | 77.704 | 844.3 | 0.092 |
| 1 oz HDPE | 10.945 | 37.0 | 0.295 |
| 12 oz HDPE | 52.88 | 420.9 | 0.125 |
| 26 oz HDPE | 81.49 | 844.3 | 0.096 |

The data in Tables 2.1-5 and Examples 2.1-5 provide stability data for samples of PEG 3350 that were stored for various time periods up to 9 months at several temperatures ranging from 25° C. to 60° C. and in containers made of different materials. Some of these tests are ongoing. These data suggest that air oxidation plays a role in the degradation of the solutions.

Samples of concentrated PEG 3350 solutions exhibit the most extensive decomposition when the samples are stored at elevated temperature in HDPE bottles. In Example 2.1, a sample of oral concentrate that was stored in a 1 ounce HDPE bottle at 40° C. for 6 months contained 422 ppm of acetaldehyde and contained only 84.6% of the PEG 3350 that was originally in the solution that was charged to the bottle (Table 2.1). These are well outside the preferred ranges of active ingredient PEG 3350 and the upper limit of acetaldehyde degradation product. Tables 2.2 and 2.3 show similar but less severe trends using larger sizes of HDPE bottles. In Table 3.2, the amount of formic acid and the extent of PEG 3350 degradation exceed the preferred limits after being stored at 40° C. for 6 months in a 1 ounce HDPE container. For comparison the samples that were stored at temperatures up to 50° C. for up to 6 months in 1.5 ounce or 12.1 ounce PET bottles (Tables 3.1 and 5) did not exhibit significant amounts of undesirable degradation products of the PEG 3350 or a significant decrease in the amount of PEG 3350.

Example 4.2 and Table 4.2 provide data on samples of the solution concentrate stored at elevated temperatures, where the concentrate samples were acidified with citric acid rather than hydrochloric acid. There were no stability issues with the PET containers, but samples in 1 ounce HDPE bottles contained up to 0.5% formic acid and exhibited extensive PEG 3350 degradation after storage at 60° C. for 3 months.

### EXAMPLES

### Example 1

The solution concentrate is made on a 2839 kg scale using the following method. Water, USP purified (1466 kg), is added to a stainless steel mixing vessel which has first been passivated by pretreatment with citric acid solution and rinsing with water. The mixing vessel is heated to 50-55° C. with continuous mixing to compensate for the cooling effect of the endothermic dissolution of the PEG 3350. Sodium benzoate (9 kg) is then added to the vessel with stirring until it is completely dissolved. PEG 3350 (1609 kg) is added at an approximate rate of 12-15 kg/minutes while continuously mixing. The solution is mixed until the PEG 3350 is completely dissolved. Hydrochloric acid 10% solution, NF (˜26 kg) is then added and the solution is mixed until it is homogeneous. The pH is adjusted to 4.0 with additional hydrochloric acid, and water is then added with mixing to bring the solution to the correct weight.

This same process has also been carried out on a laboratory scale (12.8 kg) using the same procedure in a smaller stirred vessel that has been passivated before use by rinsing three times for ten minutes with 15% citric acid solution.

### Example 2.1

Stability tests were carried out in sealed 1-ounce HDPE bottles containing solution concentrate from a 2839 kg batch of the solution concentrate. The bottles were sealed with a minimum amount of head space to minimize the amount of air in the bottles. One test was carried out at each of three different sets of conditions for the times presented in Table 2.1. Acetaldehyde (AcH) and formaldehyde were analyzed and the data are presented in the table (ppm). Acetaldehyde levels (422 ppm) greatly exceeded the proposed limits when the bottle was stored for 6 months at 40° C./20% RH, and also (at 120 ppm) exceeded the proposed limit after the bottle was stored for 6 months at 30° C./65% RH. The amount of PEG 3350 was measured and is presented as the % of PEG 3350 compared with PEG 3350 that was originally charged to the bottles. Only 84.6% of the PEG 3350 that was originally charged was still present in the bottle that was stored for 6 months at 40° C./20% RH . Ethylene glycol was measured at a level of 1200 ppm in the sample that had been kept at 40° C. Acetaldehyde was also analyzed when the solution concentrate was first mixed and was found to be present at time=zero at a level of 97 ppm. These data illustrate the degradation profile of the solution concentrate in 1 ounce HDPE bottles, which are the most oxygen permeable containers per unit volume of contents, and also support the hypothesis that degradation reactions are caused by oxygen that permeates into the vessel from the air.

TABLE 2.1
Samples in 1-Ounce HDPE Bottles

| | | | | Formaldehyde |
|---|---|---|---|---|
| Time | Temperature/RH | % PEG | AcH (ppm) | (ppm) |
| 9 mos. | 25° C./40% | 93.6% | 97 | 96 |
| 6 mos. | 30° C./65% | 93.3% | 120 | 75 |
| 6 mos. | 40° C./20% | 84.6% | 422 | 38 |

### Example 2.2

Stability tests were carried out in sealed 12.1-ounce HDPE bottles containing solution concentrate from three 2839 kg batches of the solution concentrate. The bottles were sealed with a minimum amount of head space to minimize the amount of air in the bottles. Three tests were carried out at each of three different sets of conditions for the times presented in Table 2.2. Acetaldehyde (AcH) and formaldehyde were analyzed and the data are presented as ranges measured in the table (ppm). The amount of PEG 3350 was measured and is presented as a range for the 3 tests in % of PEG 3350 compared with the PEG 3350 that was originally charged to the bottles. Ethylene glycol was not measured at the two lowest temperatures and was not detected in the samples at the highest temperature (40° C.). NQ in the table means “not quantifiable” because the amounts that were detected were too small to quantify.

Acetaldehyde was also analyzed when the solution concentrate was first mixed and was found to be present at time=zero (before stability studies began) at levels of 97 ppm, 102 ppm, and 88 ppm for the three different batches of solution concentrate. Acetaldehyde levels (257-340 ppm) greatly exceeded the proposed limits when the bottle was stored for 6 months at 40° C. 20% RH.

TABLE 2.2
Samples in 12.1-Ounce HDPE Bottles

| | | | | Formaldehyde |
|---|---|---|---|---|
| Time | Temperature/RH | % PEG | AcH (ppm) | (ppm) |
| 9 mos. | 25° C./40% | 100.3-101.3% | 70-80 | 41-55 |
| 6 mos. | 30° C./65% | 98.2-98.6% | 92-99 | 31-32 |
| 6 mos. | 40° C./20% | 95.7-96.1% | 257-340 | NQ-22 |

### Example 2.3

Stability tests were carried out in sealed 26.3-ounce HDPE bottles containing solution concentrate from three 2839 kg batches of the solution concentrate. The bottles were sealed with a minimum amount of head space to minimize the amount of air in the bottle. Three tests were carried out at each of three different sets of conditions for the times presented in Table 2.3. Acetaldehyde (AcH) and formaldehyde were analyzed, and the data are presented as the ranges that were measured in three tests (ppm). Acetaldehyde levels of 272-285 ppm greatly exceeded the proposed limits when the bottles were stored for 6 months at 40° C./20% RH. The amount of PEG 3350 was measured and is presented as a range for the 3 tests in the % of PEG 3350 that is present compared with the amount of PEG 3350 that was originally charged to the bottles. Ethylene glycol was not measured at the lowest temperature and was not detected in the samples at the highest two temperatures (30° C. and 40° C.). NQ in the table means “not quantifiable” because the amounts that were detected were too small to quantify.

Acetaldehyde was also analyzed when the solution concentrate was first mixed and was found to be present at time=zero (before stability studies began) at levels of 98 ppm, 102 ppm, and 88 ppm for the three different batches of solution concentrate. All degradants except acetaldehyde generated in the 26.3 ounce HDPE container were within the lowest proposed limits.

TABLE 2.3
Samples in 26.3-Ounce HDPE Bottles

| | Temperature/ | | | |
|---|---|---|---|---|
| Time | RH | % PEG | AcH (ppm) | Formaldehyde ppm) |
| 9 mos. | 25° C./40% | 101.4-101.8% | 58-92 | 28-30 |
| 6 mos. | 30° C./65% | 100.0-100.6% | 99-115 | 22-23 |
| 6 mos. | 40° C./20% | 97.6-98.1% | 272-285 | NQ |

### Example 3.1

Stability tests were carried out in sealed 12.1-ounce PET bottles containing solution concentrate from a laboratory scale batch (12.8 kg) of the solution concentrate. The bottles were sealed with a minimum amount of head space to minimize the amount of air in the bottles. One test was carried out at each of two different sets of conditions for 6 months. The data are presented in Table 3.1. Acetaldehyde (AcH) and formaldehyde were analyzed and the data are presented in the table (ppm). The amount of PEG 3350 was measured and is presented as the % of PEG 3350 compared with PEG 3350 that was originally charged to the bottles. Ethylene glycol and formic acid were not detected.

Acetaldehyde was also analyzed when the solution concentrate was first mixed and was found to be present at time =zero (before stability studies began) at a level of 95 ppm. Contrary to HDPE bottle data (Table 2.2), no significant changes in acetaldehyde level were observed during 6 months storage at 25° C./40% and 40° C./20%. All degradants except acetaldehyde generated in the 12.1 ounce PET bottle were within the lowest proposed limits.

TABLE 3.1
Samples in 12.1-Ounce PET Bottles

| Time | Temperature/RH | % PEG | AcH (ppm) | Formaldehyde (ppm) |
|---|---|---|---|---|
| 6 mos. | 25° C./40% | 102.0% | 88 | 23 |
| 6 mos. | 40° C./20% | 104.5% | 90 | 27 |

### Example 3.2

Stability tests were carried out in sealed HDPE bottles in 3 sizes (1, 12.1, and 26.3 ounces) containing solution concentrate from a laboratory scale batch (12.8 kg) of the solution concentrate. The bottles were sealed with a minimum amount of head space to minimize the amount of air in the bottles. One test was carried out at each of two different sets of conditions for each of the 3 sizes of bottles for 6 months. The data are presented in Table 3.2. Acetaldehyde (AcH) and formaldehyde were analyzed and the data are presented in Table 3.1 (ppm). The amount of PEG 3350 was measured and is presented as the % of PEG 3350 compared with PEG 3350 that was originally charged to the bottles. Formic acid was measured as % (w/w). NQ in the table means “not quantifiable” because the amounts that were detected were too small to quantify. The batch solution concentrate was also analyzed at time=zero (before stability tests began). The amount of PEG 3350 was measured to be 100.7% at time=zero. Acetaldehyde was present at a level of 79 ppm. Formaldehyde was not detected. Acetaldehyde was outside the proposed limits in all 3 sizes of bottles that were tested at 40° C./20% RH. Formic acid was outside the proposed limits in the 1 ounce bottle that was tested at 40° C./20% RH. The amount of PEG 3350 that was still present was outside the limits after standing for 6 months at 40° C./20% RH in the 1 ounce bottle

TABLE 3.2
Samples in HDPE Bottles - 6 Month Tests

| Bottle | Temp/ | | AcH | Formaldehyde | Formic |
|---|---|---|---|---|---|
| Size | RH | % PEG | (ppm) | (ppm) | Acid % (w/w) |
| 1 ounce | 25° C./ | 95.9% | 44 | 151 | 0.09% |
| | 40% | | | | |
| 1 ounce | 40° C./ | 85.7% | 131 | 144 | 0.33% |
| | 20% | | | | |
| 12.1 ounce | 25° C./ | 98.1% | 62 | 70 | NQ |
| | 40% | | | | |
| 12.1 ounce | 40° C./ | 95.1% | 141 | 67 | 0.10% |
| | 20% | | | | |
| 26.3 ounce | 25° C./ | 98.1% | 70 | 49 | NQ |
| | 40% | | | | |
| 26.3 ounce | 40° C./ | 96.3% | 130 | 53 | NQ |
| | 20% | | | | |

### Example 4.1

A 1.5 kg laboratory scale batch of PEG 3350 solution concentrate containing citric acid in place of hydrochloric acid was prepared by the following procedure. Water (675 g) was added to a reactor and was heated to 45° C. with stirring. Sodium benzoate (2.835 g) was then added with stirring, followed by citric acid (14.48 g) also with stirring. Finally PEG 3350 (787 g) was added. Stirring was continued, and the temperature was allowed to decrease to ambient room temperature.

### Example 4.2

Stability tests were carried out in sealed bottles made of PET, HDPE or glass using the solution concentrate containing citric acid that was made in the previous example. The bottles were sealed with a minimum amount of head space to minimize the amount of air in the bottles. Samples of the solution concentrate containing citric acid were placed in 1-ounce HDPE bottles, 1.5-ounce PET bottles, and 1-ounce glass bottles. Bottles were stored at 60° C. for 1 month and 3 months and were tested for degradation products at the end of the 1-month and 3-month storage periods. A sample of the freshly made solution was also analyzed at time=zero as a control without being stored in a bottle. The amounts of acetaldehyde (AcH), formaldehyde, and formic acid were analyzed by chromatography, and the amounts that were measured are presented in the table. Acetaldehyde and formaldehyde are presented as ppm RTA (relative to active), and formic acid is presented as % of the solution (w/w). The amount of PEG 3350 was measured and is presented as the % of PEG 3350 that is present after the test compared with the amount of PEG 3350 that was originally charged to the bottles. Ethylene glycol was not measured in these experiments. NQ in the table means “not quantifiable” because the amounts that were detected were too small to quantify. ND means that a compound was “not detected.” When the solution concentrate was formulated with citric acid instead of hydrochloric acid, all degradants generated in the 1.5 ounce PET container were within the proposed limits except for the acetaldehyde in after 1 month storage at 60° C. Also, the solution made with citric acid stored in 1 ounce HDPE bottles generated acetaldehyde and formic acid to levels above the proposed limits, and the PEG degraded excessively.

TABLE 4.2
Samples Containing Citric Acid

| | | | | | | | Formic |
|---|---|---|---|---|---|---|---|
| Bottle | | | | | | Formal- | Acid |
| Size/ | | Temp/ | | % | AcH | dehyde | % |
| Composition | | RH | Time | PEG | (ppm) | (ppm) | (w/w) |
| Control | | NA | N/A | 101.1 | 61 | ND | ND |
| 1 | oz HDPE | 60° C. | 1 month | 91.8 | 158 | 68 | 0.2% |
| 1.5 | oz PET | 60° C. | 1 month | 99.1 | 110 | NQ | ND |
| 1 | oz glass | 60° C. | 1 month | 96.0 | 117 | NQ | ND |
| 1 | oz HDPE | 60° C. | 3 months | 82.6 | 127 | 118 | 0.5% |
| 1.5 | oz PET | 60° C. | 3 months | 104.6 | 39 | 57 | 0.1% |
| 1 | oz glass | 60° C. | 3 months | 100.5 | 154 | NQ | ND |

### Example 4.3

Stability tests were carried out in sealed bottles made of HDPE using the solution concentrate containing citric acid that was made in the previous example. The bottles were sealed with a minimum amount of head space to minimize the amount of air in the bottles. Samples of the solution concentrate containing citric acid were placed in 1-ounce HDPE bottles. Bottles were stored at 25° C./ambient humidity and 40° C./ambient humidity for 6 months and were tested for degradation products at predetermined time intervals. A sample of the freshly made solution was also analyzed at time=zero as a control without being stored in a bottle. Acetaldehyde and formaldehyde are presented as ppm RTA (relative to active). Ethylene glycol and formic acid are presented as ppm and % w/w of the solution, respectively. The amount of PEG 3350 was measured and is presented as the % of PEG 3350 that is present after the test compared with the amount of PEG 3350 that was originally charged to the bottles. NQ in the table means “not quantifiable” because the amounts that were detected were too small to quantify. ND means that a compound was “not detected.” When the solution concentrate was formulated with citric acid instead of hydrochloric acid, formaldehyde and ethylene glycol were outside the proposed limit for the bottles at all storage conditions. Formic acid increased over time but met the proposed limit. The amount of PEG 3350 that was still present was outside the limits after standing for 6 months at 40° C./ambient humidity.

TABLE 4.3
Samples Containing Citric Acid

| | | | | | | Formic | |
|---|---|---|---|---|---|---|---|
| Bottle | Temp/ | | | AcH | Formaldehyde | Acid % | Ethylene |
| Size/Composition | RH | Time | % PEG | (ppm) | (ppm) | (w/w) | Glycol (ppm) |
| Control | NA | N/A | 101.0 | 86 | NQ | ND | ND |
| 1 oz HDPE | 25° C. | 6 month | 97.1 | 52 | 105 | 0.1 | 460 |
| 1 oz HDPE | 40° C. | 3 month | 98.6 | 70 | 86 | 0.2 | 592 |
| 1 oz HDPE | 40° C. | 6 month | 89.6 | 63 | 94 | 0.3 | 1151 |

### Example 5

Stability tests were carried out in sealed PET bottles in 2 sizes (1.5 and 12.1 ounces) containing solution concentrate from a laboratory scale batch (12.8 kg) of the solution concentrate. The bottles were sealed with a minimum amount of head space to minimize the amount of air in the bottles. Stability tests were carried out at three different sets of conditions for each of the 2 sizes of bottles after 3 or 6 months. The data are presented in Table 5. Acetaldehyde (AcH) and formaldehyde were analyzed and the data are presented in the table (ppm). The amount of PEG 3350 was measured and is presented as the % of PEG 3350 compared with PEG 3350 that was originally charged to the bottles. All of the samples were tested for formic acid, and all but two of the samples were tested for ethylene glycol. Formic acid and ethylene glycol were not detected in any of the samples that were tested. NQ in the table means “not quantifiable” because the amounts that were detected were too small to quantify.

The composition of the solution concentrate was also measured before the stability studies began, as follows: PEG, 99.2%; formaldehyde, NQ; acetaldehyde, 95 ppm; and formic acid, not detected.

The samples in the 1.5 oz PET bottles that had been subjected to storage at the 3 different temperatures were measured for dissolved oxygen at the end of the 6-month trial. The largest amount of dissolved oxygen in any of the 3 samples was 2.04 ppm. All degradants except acetaldehyde generated in the 1.5 ounce and 12.1 ounce PET containers at the various temperatures were within the proposed limits. As expected, the level of acetaldehyde in the 1.5 ounce PET containers were higher than that in the 12.1 ounce PET containers under same stability conditions.

TABLE 5
Stability Studies in PET Bottles

| Bottle | | | | | Formaldehyde |
|---|---|---|---|---|---|
| Size | Temp | Time | % PEG | AcH (ppm) | (ppm) |
| 12.1 oz. | 25° C. | 1-6 months | 98.8-102.0 | 88-95 | NQ-23 |
| 12.1 oz. | 40° C. | 1-6 months | 99.7-104.5 | 90-96 | NQ-27 |
| 12.1 oz. | 50° C. | 1-3 months | 100.1-103.3 | 95-98 | NQ-24 |
| 1.5 oz. | 25° C. | 1-6 months | 100.7-103.1 | 77-85 | NQ-24 |
| 1.5 oz. | 40° C. | 1-6 months | 100.7-106.9 | 91-116 | NQ |
| 1.5 oz. | 50° C. | 1-3 months | 101.9-105.7 | 124-140 | NQ |

### Example 6

The transmission rates of oxygen into bottles were measured at atmospheric pressure, 23° C., and 0% relative humidity (RH), where the air contained 21% oxygen. The permeability was also measured for pure oxygen under the same conditions. The tests were carried out on 12.1 ounce and 26.3 ounce bottles made of HDPE and PET. The data are shown in Table 6 below.

TABLE 6
Oxygen Transmission Rates into Bottles

| Oxygen | | | | | | |
|---|---|---|---|---|---|---|
| Transmission | | | | | | |
| Rate | 1 oz. | 1.5 oz. | 12.1 oz. | 12.1 oz. | 26.3 oz. | 26.3 oz. |
| (cc/bottle/day) | HDPE | PET | HDPE | PET | HDPE | PET |
| 23° C., | 0.12 | 0.02 | 0.49 | 0.04 | 0.56 | 0.06 |
| 0% RH, 21% | | | | | | |
| Oxygen | | | | | | |
| 23° C., | 0.59 | 0.09 | 2.31 | 0.21 | 2.68 | 0.27 |
| 0% RH, 100% | | | | | | |
| Oxygen | | | | | | |

It can be seen that PET is better as an oxygen barrier than HDPE by a factor of about 10 for each bottle size. Flat sheet PET has an oxygen transmission rate of 6.5-13 cc-mil /100 in²-day, whereas flat sheet HDPE has an oxygen transmission rate of 185-260 cc-mil /100 in²-day, so that flat sheet PET is better than flat sheet HDPE as an oxygen barrier by a factor of about 20-30. The barrier properties of PET compared with HDPE are not as great for bottles (a factor of about 10) as for flat sheet plastic (a factor of about 20-30), but the difference is still large enough to have a significant effect on the amount of oxygen that permeates into an HDPE bottle compared with a PET bottle. The transmission rate data described above were obtained using ASTM F-1307-02 Standard Test Method for Oxygen Transmission Rate Through Dry Packages Using a Coulometric Sensor Test. Although the transmission rate data were obtained using dry bottles, the test data on solutions in sealed bottles follow the same trends in degradation as are expected based on the oxygen transmission rates in dry bottles.

### Example 7

Twelve samples of PEG 3350 from a specific vendor (BASF) were formulated into small batches of solution concentrate which were acidified with hydrochloric acid to analyze for acetaldehyde that was formed when the formulations were initially manufactured. The amount of acetaldehyde that formed in the 12 example formulations during the manufacturing process ranged from 36-51 ppm. For comparison, batches of solution concentrate that were made previously to these tests using PEG 3350 from other sources and that were tested for initial acetaldehyde levels had levels of acetaldehyde ranging from 79-102 ppm. These data demonstrate the feasibility of setting a limit of 60 ppm acetaldehyde on incoming PEG 3350.

### Example 7.1

Stability tests were carried out in sealed 12 oz PET bottle containing solution concentrate containing PEG 3350 from BASF (7 kg scale batch). The bottles were sealed with a minimum amount of head space to minimize the amount of air in the bottles. Stability tests were carried out at two different sets of conditions over 6 months. The data are presented in Table 7. Acetaldehyde (AcH) and formaldehyde were analyzed and the data are presented in the table (ppm). The amount of PEG 3350 was measured and is presented as the % of PEG 3350 compared with PEG 3350 that was originally charged to the bottles. All of the samples were tested for formic acid, ethylene glycol, and diethylene glycol. NQ in the table means “not quantifiable” because the amounts that were detected were too small to quantify. As presented in Table 8, formaldehyde was below quantifiable level during 6 months storage at 25° C./40% and 40° C./20%. Acetaldehyde generated for the samples at all storage conditions was within the proposed limit although its level moderately increased with time and temperature. Ethylene glycol and diethylene glycol were not detected in any of the samples that were tested.

TABLE 7
Stability Study in PET bottle

| | | | AcH | Formaldehyde | Formic Acid |
|---|---|---|---|---|---|
| Temp/RH | Time | % PEG | (ppm) | (ppm) | % (w/w) |
| NA | Initial | 102.9 | 22 | ND | ND |
| 25° C./40% RH | 1 month | 101.7 | 29 | ND | ND |
| 40° C./20% RH | 1 month | 102.2 | 28 | NQ | ND |
| 25° C./40% RH | 3 month | 101.9 | 31 | NQ | ND |
| 40° C./20% RH | 3 months | 102.7 | 35 | NQ | ND |
| 25° C./40% RH | 6 months | 102.2 | 34 | NQ | ND |
| 40° C./20% RH | 6 months | 103.5 | 41 | NQ | ND |

### Example 8

### Example 8.1

Formulations were prepared according to the invention comprising Editate Disodium (EDTA) to determine its potential effect on further minimizing the degradation products of PEG 3350 solution. Formulations were prepared as follows: Water USP was added to an appropriate mixing vessel equipped with a propeller and heated to 50-60° C. EDTA USP was added and mixed until fully dissolved. This solution was then cooled to 40° C. and Sodium Benzoate, NF was added and mixed until fully dissolved. The solution was then maintained at 40° C. while adding PEG 3350 slowly and mixing continued until completely dissolved. At this point the heater was turned off and the solution allowed to cool to room temperature with continued mixing. Next, Hydrochloric Acid Diluted was added and mixed well. The pH of the solution was maintained between 4.0-4.3 using Hydrochloric Acid Diluted (q.s. part) and Water USP added q.s. the batch while mixing an additional 10 minutes to ensure uniformity. The mixing speed was decreased in this final step to deaerate the final product. The final pH is measured to ensure pH between 4.0-4.3.

Laboratory scale batches (˜10 kg) of formulations containing no EDTA and four different EDTA levels were manufactured using the same lot of PEG 3350 (BASF) and packaged into 12 oz PET bottles. The closure for all the PET bottles were applied using a torque meter (Shimpo) with the application torque between 15 and 25 in/lb. The closure foils were induction sealed at a setting of “low” for 5 seconds. The packaged samples were then placed in the stability chambers maintained at different temperatures. At predetermined time intervals, one sample from each storage condition was tested for PEG 3350 and its degradation products. The batch of the formula without EDTA was manufactured using the same lot of PEG 3350 and then packaged into 12 oz PET bottles. These packaged samples were subjected to accelerated informal stability studies. Table 8.1 lists the composition for the tested formulations.

TABLE 8.1
Formula Comparison for Formulations Containing EDTA

| | Ex 8A | Ex. 8B | Ex. 8C | Ex. 8D | Ex. 8E |
|---|---|---|---|---|---|
| | pH = 4.1 | pH = 4.0 | pH = 4.1 | pH = 4.2 | pH = 4.2 |
| PEG 3350, USP | 56.67 | 56.67 | 56.67 | 56.67 | 56.67 |
| Edetate Disodium, | — | 0.025 | 0.05 | 0.075 | 0.10 |
| USP | | | | | |
| Sodium Benzoate, | 0.33 | 0.33 | 0.33 | 0.33 | 0.33 |
| NF | | | | | |
| Hydrochloric | 0.95 | 0.95 | 0.95 | 0.95 | 0.95 |
| Acid, Diluted, NF | | | | | |
| Water, USP | 51.6 | 50.0 | 50.0 | 50.0 | 50.0 |
| Purified | | | | | |
| Hydrochloric | q.s. | q.s. | q.s. | q.s. | q.s. |
| Acid, Diluted, NF* | | | | | |
| Water, USP | q.s. | q.s. | q.s. | q.s. | q.s. |
| Purified | | | | | |

*Used for pH adjustment if necessary

Accelerated stability data obtained on the formulations containing no EDTA and formulations containing different levels of EDTA are presented in Table 8.2 through Table 8.6. All levels of EDTA studied markedly reduced acetaldehyde level as compared to the control after 12 weeks at higher temperatures (>40° C.). The lower level (0.025% w/v) of EDTA was enough to curtail acetaldehyde degradation process in the samples at all storage conditions. A slight increase in formaldehyde level was also observed for the formulations containing EDTA after 4 weeks at 60° C. but was well blow the lowest proposed specification limit. No quantifiable levels of Diethylene Glycol, Ethylene Glycol, and Formic Acid were observed in any of the samples studied.

TABLE 8.2
Accelerated Stability Results for Formula Ex 8A

| | Time (weeks)/Storage Condition | | | | | | | | | | | | | | | | | |
|---|---|---|---|---|---|---|---|---|---|---|---|---|---|---|---|---|---|---|
| | | 2 | | | 4 | | | 6 | | | | 8 | | 10 | | 12 | | |
| Test | Int | 40 | 50 | 60 | 40 | 50 | 60 | 25H | 40 | 50 | 60 | 40 | 50 | 40 | 50 | 25H | 40 | 50 |
| Assay - PEG 3350 | 103.1 | NT | 104.0 | 103.9 | 102.4 | 103.8 | 104.1 | 102.7 | NT | 107.0 | 103.2 | NT | 108.6 | NT | 102.9 | 102.4 | 102.4 | 103.2 |
| Assay - Formaldehyde | ND | NT | NQ | ND | ND | ND | ND | ND | NT | ND | ND | NT | ND | NT | ND | NQ | ND | ND |
| Assay - Acetaldehyde | 30 | NT | 30 | 75 | 29 | 48 | 98 | 26 | NT | 62 | 125 | NT | 72 | NT | 80 | 28 | 50 | 89 |
| Assay - Diethylene | ND | NT | ND | ND | ND | ND | ND | ND | NT | ND | ND | NT | ND | NT | ND | ND | ND | ND |
| Glycol (DEG) | | | | | | | | | | | | | | | | | | |
| Assay - Ethylene | ND | NT | ND | ND | ND | ND | ND | ND | NT | ND | ND | NT | ND | NT | ND | ND | ND | ND |
| Glycol (EG) | | | | | | | | | | | | | | | | | | |
| Assay - Combination | ND | NT | ND | ND | ND | ND | ND | ND | NT | ND | ND | NT | ND | NT | ND | ND | ND | ND |
| of DEG + EG | | | | | | | | | | | | | | | | | | |
| Assay - Formic acid | ND | NT | ND | ND | ND | ND | ND | ND | NT | ND | ND | NT | ND | NT | ND | ND | ND | ND |
| Assay - other acids** | NT | NT | NT | NT | NT | NT | NT | NT | NT | NT | NT | NT | NT | NT | NT | NT | NT | NT |
| Assay - Tri- and | ND | NT | ND | ND | ND | ND | ND | ND | NT | ND | ND | NT | ND | NT | ND | ND | ND | ND |
| Tetra- Glycols | | | | | | | | | | | | | | | | | | |
| Assay - Penta - | ND | NT | ND | ND | ND | ND | ND | ND | NT | ND | ND | NT | ND | NT | ND | ND | ND | ND |
| through Octa- Glycols | | | | | | | | | | | | | | | | | | |
| Total Related | ND | NT | ND | ND | ND | ND | ND | ND | NT | ND | ND | NT | ND | NT | ND | ND | ND | ND |
| Substances | | | | | | | | | | | | | | | | | | |

25H: 25° C./60% RH (Relative Humidity),
40: 40° C./ambient humidity,
50: 50° C./ambient humidity,
60: 60° C./ambient humidity,
Int: Initial,
LS: Label Strength,
RTA: Relative to Active,
NQ: Not Quantifiable,
ND: None Detected,
NMT: Not More Than,
NT: Not Tested
**Acetic acid, Oxalic acid, Glyoxylic acid, Malonic acid, and Lactic acid

TABLE 8.3
Accelerated Stability Results for Formula Ex. 8B

| | Time (Weeks)/Storage Condition | | | | | | | | | | | | | | | | | |
|---|---|---|---|---|---|---|---|---|---|---|---|---|---|---|---|---|---|---|
| | | 2 | | | 4 | | | 6 | | | | 8 | | 10 | | 12 | | |
| Test | Int | 40 | 50 | 60 | 40 | 50 | 60 | 25H | 40 | 50 | 60 | 40 | 50 | 40 | 50 | 25H | 40 | 50 |
| Assay - PEG 3350 | NT | NT | NT | NT | NT | NT | NT | 102.8 | 102.1 | 103.1 | 103.1 | NT | NT | NT | NT | 104.4 | 104.7 | 103.8 |
| Assay - | ND | ND | NQ | NQ | NQ | NQ | NQ | ND | NQ | NQ | 20.0 | NQ | NQ | NQ | NQ | NQ | 20 | 20 |
| Formaldehyde | | | | | | | | | | | | | | | | | | |
| Assay - | 28.1 | NQ | NQ | 21.3 | 27.5 | 28.4 | 30.8 | 21.3 | 22.4 | 23.5 | 25.3 | 29 | 30 | 29 | 30 | 27 | 28 | 30 |
| Acetaldehyde | | | | | | | | | | | | | | | | | | |
| Assay - Diethylene | NT | NT | NT | NT | NT | NT | NT | D | ND | ND | ND | ND | ND | ND | ND | ND | ND | ND |
| Glycol (DEG) | | | | | | | | | | | | | | | | | | |
| Assay - Ethylene | NT | NT | NT | NT | NT | NT | NT | ND | ND | ND | ND | ND | ND | ND | ND | ND | ND | ND |
| Glycol (EG) | | | | | | | | | | | | | | | | | | |
| Assay - | NT | NT | NT | NT | NT | NT | NT | ND | ND | ND | ND | ND | ND | ND | ND | ND | ND | ND |
| Combination of | | | | | | | | | | | | | | | | | | |
| DEG + EG | | | | | | | | | | | | | | | | | | |
| Assay - Formic acid | NT | NT | NT | NT | NT | NT | NT | ND | ND | ND | ND | ND | ND | NQ | NQ | ND | NQ | NQ |
| Assay - other acids* | NT | NT | NT | NT | NT | NT | NT | NQ¹ | ND | NQ² | ND | NT | NT | NT | NT | NT | NT | NT |
| Assay - Tri- and | NT | NT | NT | NT | NT | NT | NT | NT | NT | NT | NT | NT | NT | NT | NT | NT | NT | NT |
| Tetra- Glycols | | | | | | | | | | | | | | | | | | |
| Assay - Penta - | NT | NT | NT | NT | NT | NT | NT | NT | NT | NT | NT | NT | NT | NT | NT | ND | ND | ND |
| through Octa- | | | | | | | | | | | | | | | | | | |
| Glycols | | | | | | | | | | | | | | | | | | |

25H: 25° C./60% RH (Relative Humidity),
40: 40° C./ambient humidity,
50: 50° C./ambient humidity,
60: 60° C./ambient humidity,
Int: Initial,
LS: Label Strength,
RTA: Relative to Active,
NQ: Not Quantifiable,
ND: None Detected,
NMT: Not More Than,
NT: Not Tested
*Acetic acid, Oxalic acid, Glyoxylic acid, Malonic acid, and Lactic acid,
¹NQ for Acetic acid, ND for the other acids,
²NQ for Acetic acid and Oxalic Acid,
ND for the other acids

TABLE 8.4
Accelerated Stability Results for Formula Ex 8C

| | Time (Weeks)/Storage Condition | | | | | | | | | | | | | | |
|---|---|---|---|---|---|---|---|---|---|---|---|---|---|---|---|
| | | 2 | | | 4 | | | 6 | | | | 8 | | 10 | |
| Test | Int | 40 | 50 | 60 | 40 | 50 | 60 | 25H | 40 | 50 | 60 | 40 | 50 | 40 | 50 |
| Assay - PEG 3350 | NT | NT | NT | NT | NT | NT | NT | 102.8 | 102.5 | 102.9 | 103.3 | NT | NT | NT | NT |
| Assay - Formaldehyde | ND | ND | NQ | NQ | NQ | NQ | NQ | ND | NQ | NQ | 20.4 | NQ | NQ | NQ | 20 |
| Assay - Acetaldehyde | 27.2 | 20.9 | NQ | 19.9 | 27.3 | 28.5 | 30.1 | 21.9 | 22.3 | 24.2 | 25.5 | 29 | 30 | 28 | 29 |
| Assay - Diethylene Glycol | NT | NT | NT | NT | NT | NT | NT | ND | ND | ND | ND | ND | ND | ND | ND |
| (DEG) | | | | | | | | | | | | | | | |
| Assay - Ethylene Glycol | NT | NT | NT | NT | NT | NT | NT | ND | ND | ND | ND | ND | ND | ND | ND |
| (EG) | | | | | | | | | | | | | | | |
| Assay - Combination of | NT | NT | NT | NT | NT | NT | NT | ND | ND | ND | ND | ND | ND | ND | ND |
| DEG + EG | | | | | | | | | | | | | | | |
| Assay - Formic acid | NT | NT | NT | NT | NT | NT | NT | ND | ND | ND | ND | ND | ND | NQ | NQ |
| Assay - other acids* | NT | NT | NT | NT | NT | NT | NT | ND | ND | NQ¹ | ND | NT | NT | NT | NT |
| Assay - Tri- and Tetra- | NT | NT | NT | NT | NT | NT | NT | NT | NT | NT | NT | NT | NT | NT | NT |
| Glycols | | | | | | | | | | | | | | | |
| Assay - Penta - through | NT | NT | NT | NT | NT | NT | NT | NT | NT | NT | NT | NT | NT | NT | NT |
| Octa- Glycols | | | | | | | | | | | | | | | |

25H: 25° C./60% RH (Relative Humidity),
40: 40° C./ambient humidity,
50: 50° C./ambient humidity,
60: 60° C./ambient humidity,
Int: Initial,
LS: Label Strength,
RTA: Relative to Active,
NQ: Not Quantifiable,
ND: None Detected,
NMT: Not More Than,
NT: Not Tested
*Acetic acid, Oxalic acid, Glyoxylic acid, Malonic acid, and Lactic acid,
¹NQ for Oxalic Acid,
ND for the other acids

TABLE 8.5
Accelerated Stability Results for Formula Ex. 8D

| | Time (Weeks)/Storage Condition | | | | | | | | | | | | | | |
|---|---|---|---|---|---|---|---|---|---|---|---|---|---|---|---|
| | | 2 | | | 4 | | | 6 | | | | 8 | | 10 | |
| Test | Int | 40 | 50 | 60 | 40 | 50 | 60 | 25H | 40 | 50 | 60 | 40 | 50 | 40 | 50 |
| Assay - PEG 3350 | NT | NT | NT | NT | NT | NT | NT | 100.2 | 102.7 | 100.5 | 102.9 | NT | NT | NT | NT |
| Assay - Formaldehyde | ND | NQ | NQ | 20.4 | NQ | NQ | 20.8 | ND | NQ | NQ | 22.1 | NQ | 22 | 21 | 22 |
| Assay - Acetaldehyde | 27.8 | NQ | NQ | 20.4 | 27.4 | 27.9 | 29.6 | 20.9 | 23.6 | 22.6 | 23.8 | 29 | 30 | 28 | 29 |
| Assay - Diethylene Glycol | NT | NT | NT | NT | NT | NT | NT | ND | ND | ND | ND | ND | ND | ND | ND |
| (DEG) | | | | | | | | | | | | | | | |
| Assay - Ethylene Glycol | NT | NT | NT | NT | NT | NT | NT | ND | ND | ND | ND | ND | ND | ND | ND |
| (EG) | | | | | | | | | | | | | | | |
| Assay - Combination of | NT | NT | NT | NT | NT | NT | NT | ND | ND | ND | ND | ND | ND | ND | ND |
| DEG + EG | | | | | | | | | | | | | | | |
| Assay - Formic acid | NT | NT | NT | NT | NT | NT | NT | ND | ND | ND | ND | ND | ND | NQ | NQ |
| Assay - other acids* | NT | NT | NT | NT | NT | NT | NT | ND | ND | NQ¹ | ND | NT | NT | NT | NT |
| Assay - Tri- and Tetra- | NT | NT | NT | NT | NT | NT | NT | NT | NT | NT | NT | NT | NT | NT | NT |
| Glycols | | | | | | | | | | | | | | | |
| Assay - Penta - through | NT | NT | NT | NT | NT | NT | NT | NT | NT | NT | NT | NT | NT | NT | NT |
| Octa- Glycols | | | | | | | | | | | | | | | |

25H: 25° C./60% RH (Relative Humidity),
40: 40° C./ambient humidity,
50: 50° C./ambient humidity,
60: 60° C./ambient humidity,
Int: Initial,
LS: Label Strength,
RTA: Relative to Active,
NQ: Not Quantifiable,
ND: None Detected,
NMT: Not More Than,
NT: Not Tested
*Acetic acid, Oxalic acid, Glyoxylic acid, Malonic acid, and Lactic acid,
¹NQ for Oxalic Acid,
ND for the other acids

TABLE 8.6
Accelerated Stability Results for Formula Ex. 8E

| | Time/58 (Weeks)/Storage Condition | | | | | | | | | | | | | | |
|---|---|---|---|---|---|---|---|---|---|---|---|---|---|---|---|
| | | 2 | | | 4 | | | 6 | | | | 8 | | 10 | |
| Test | Int | 40 | 50 | 60 | 40 | 50 | 60 | 25H | 40 | 50 | 60 | 40 | 50 | 40 | 50 |
| Assay - PEG 3350 | NT | NT | NT | NT | NT | NT | NT | 102.0 | 101.8 | 101.1 | 102.1 | NT | NT | NT | NT |
| Assay - Formaldehyde | ND | NQ | NQ | 21.7 | NQ | 20.3 | 23.8 | NQ | NQ | 21.7 | 25.8 | 20 | 22 | 23 | 25 |
| Assay - Acetaldehyde | 27.7 | NQ | NQ | 20.2 | 27.5 | 28.3 | 29.3 | 22.3 | 22.9 | 23.5 | 23.6 | 29 | 30 | 29 | 29 |
| Assay - Diethylene Glycol | NT | NT | NT | NT | NT | NT | NT | ND | ND | ND | ND | ND | ND | ND | ND |
| (DEG) | | | | | | | | | | | | | | | |
| Assay - Ethylene Glycol | NT | NT | NT | NT | NT | NT | NT | ND | ND | ND | ND | ND | ND | ND | ND |
| (EG) | | | | | | | | | | | | | | | |
| Assay - Combination of | NT | NT | NT | NT | NT | NT | NT | ND | ND | ND | ND | ND | ND | ND | ND |
| DEG + EG | | | | | | | | | | | | | | | |
| Assay - Formic acid | NT | NT | NT | NT | NT | NT | NT | ND | ND | ND | ND | ND | ND | NQ | NQ |
| Assay - other acids* | NT | NT | NT | NT | NT | NT | NT | ND | ND | NQ¹ | ND | NT | NT | NT | NT |
| Assay - Tri- and Tetra- | NT | NT | NT | NT | NT | NT | NT | NT | NT | NT | NT | NT | NT | NT | NT |
| Glycols | | | | | | | | | | | | | | | |
| Assay - Penta - through | NT | NT | NT | NT | NT | NT | NT | NT | NT | NT | NT | NT | NT | NT | NT |
| Octa- Glycols | | | | | | | | | | | | | | | |

25H: 25° C./60% RH (Relative Humidity),
40: 40° C./ambient humidity,
50: 50° C./ambient humidity,
60: 60° C./ambient humidity,
Int: Initial,
LS: Label Strength,
NQ: Not Quantifiable,
ND: None Detected,
NMT: Not More Than,
NT: Not Tested
*Acetic acid, Oxalic acid, Glyoxylic acid, Malonic acid, and Lactic acid,
¹NQ for Oxalic Acid,
ND for the other acids

### Example 8.2

An additional stability study was conducted with formulations containing lower levels (0.01 and 0.02% w/v) of EDTA to assess the effects of lower concentrations of EDTA to effectively inhibit the degradation process of PEG 3350. Formulations containing the lower levels were prepared and bottled in PET according to methods described in Example 8.1. As shown in Tables 8.7 and 8.8, the lower levels of EDTA still provide for non-quantifiable or non-detectable levels of Diethylene Glycol, Ethylene Glycol, and Formic Acid and non-quantifiable levels of formaldehyde while maintaining levels of acetaldehyde well below the lowest proposed specification limit.

TABLE 8.7
Accelerated Stability Results for Formulation Containing EDTA 0.02% w/w

| | | Time (Weeks)/Storage Condition | | | | | | |
|---|---|---|---|---|---|---|---|---|
| | Test | | 2 | | | 4 | | |
| Test | Specification | Int | 40 | 50 | 60 | 40 | 50 | 60 |
| Specific Gravity | Report | 1.094 | NT | NT | NT | NT | NT | NT |
| Assay - PEG 3350 | 90.0-110.0% | 94.8 | NT | NT | NT | NT | NT | NT |
| | LS | | | | | | | |
| Assay - Formaldehyde | NMT 70 ppm RTA | ND | NQ | NQ | NQ | NQ | NQ | NQ |
| Assay - Acetaldehyde | NMT 70 ppm | 27 | 26 | 27 | 29 | 26 | 26 | 29 |
| | RTA | | | | | | | |
| Assay - Diethylene Glycol | NMT 180 ppm | ND | ND | ND | ND | ND | ND | ND |
| (DEG) | | | | | | | | |
| Assay - Ethylene Glycol (EG) | NMT 180 ppm | ND | ND | ND | ND | ND | ND | ND |
| Assay - Combination of DEG + | NMT 360 ppm | ND | ND | ND | ND | ND | ND | ND |
| EG | | | | | | | | |
| Assay - Formic acid | NMT 0.3% | ND | NT | NT | NT | NT | NT | NT |
| Assay - other acids* | NMT 0.3% for the | NQ¹ | NT | NT | NT | NT | NT | NT |
| | each acid | | | | | | | |
| Assay - Tri- and Tetra- | Report | ND | NT | NT | NT | NT | NT | NT |
| Glycols | | | | | | | | |
| Assay - Penta - through Octa- | Report | ND | NT | NT | NT | NT | NT | NT |
| Glycols | | | | | | | | |
| Total Related Substances | NMT 1.0% | NQ | NT | NT | NT | NT | NT | NT |

25H: 25° C./60% RH (Relative Humidity),
40: 40° C./ambient humidity,
50: 50° C./ambient humidity,
60: 60° C./ambient humidity,
Int: Initial,
LS: Label Strength,
RTA: Relative to Active,
NQ: Not Quantifiable,
ND: None Detected,
NMT: Not More Than,
NT: Not Tested
*Acetic acid, Oxalic acid, Glyoxylic acid, Malonic acid, and Lactic acid,
¹NQ for Acetic Acid,
ND for the other acids

TABLE 8.8
Accelerated Stability Results for Formulation Containing EDTA 0.01% w/w

| | | Time (Weeks)/Storage Condition | | | | | | |
|---|---|---|---|---|---|---|---|---|
| | Test | | 2 | | | 4 | | |
| Test | Specification | Int | 40 | 50 | 60 | 40 | 50 | 60 |
| Specific Gravity | Report | 1.095 | NT | NT | NT | NT | NT | NT |
| Assay - PEG 3350 | 90.0-110.0% | 94.8 | NT | NT | NT | NT | NT | NT |
| | LS | | | | | | | |
| Assay - Formaldehyde | NMT 70 ppm RTA | ND | ND | NQ | NQ | NQ | NQ | NQ |
| Assay - Acetaldehyde | NMT 70 ppm | 26 | 28 | 26 | 29 | 25 | 26 | 27 |
| | RTA | | | | | | | |
| Assay - Diethylene Glycol | NMT 180 ppm | ND | ND | ND | ND | ND | ND | ND |
| (DEG) | | | | | | | | |
| Assay - Ethylene Glycol | NMT 180 ppm | ND | ND | ND | ND | ND | ND | ND |
| (EG) | | | | | | | | |
| Assay - Combination of | NMT 360 ppm | ND | ND | ND | ND | ND | ND | ND |
| DEG + EG | | | | | | | | |
| Assay - Formic acid | NMT 0.3% | ND | NT | NT | NT | NT | NT | NT |
| Assay - other acids* | NMT 0.3% for the | NQ¹ | NT | NT | NT | NT | NT | NT |
| | each acid | | | | | | | |
| Assay - Tri- and Tetra- | Report | ND | NT | NT | NT | NT | NT | NT |
| Glycols | | | | | | | | |
| Assay - Penta - through | Report | ND | NT | NT | NT | NT | NT | NT |
| Octa- Glycols | | | | | | | | |
| Total Related Substances | NMT 1.0% | NQ | NT | NT | NT | NT | NT | NT |

25H: 25° C./60% RH (Relative Humidity),
40: 40° C./ambient humidity,
50: 50° C./ambient humidity,
60: 60° C./ambient humidity,
Int: Initial,
LS: Label Strength,
RTA: Relative to Active,
NQ: Not Quantifiable,
ND: None Detected,
NMT: Not More Than,
NT: Not Tested
*Acetic acid, Oxalic acid, Glyoxylic acid, Malonic acid, and Lactic acid,
¹NQ for Acetic Acid,
ND for the other acids

1. A solution concentrate, comprising (w/w) (a) 37%-57% water; (b) 0.05-0.36% sodium benzoate; (c) 42%-62% polyethylene glycol having an average molecular weight of 2680-4020 Daltons; and (d) a pharmaceutically acceptable acid in an amount sufficient to yield a solution having a pH at 25° C. in the range 3.0-5.0; wherein said solution concentrate is packaged in a sealed container having an oxygen transmission rate into the dry container no greater than 0.1 cc/container/day from air at one atmosphere of pressure and 25° C. as measured using ASTM F-1307-02.

2. The solution concentrate of claim 1, comprising (w/w) (a) 42%-52% water; (b) 0.24-0.36% sodium benzoate; and (c) 47%-57% polyethylene glycol having an average molecular weight of 3015-3685 Daltons, wherein the pH of the solution is 3.5-5.0.

3. The solution concentrate of claim 2, wherein the polyethylene glycol has an average molecular weight of 3350 Daltons.

4. The solution concentrate of claim 3, comprising about (w/w) (a) 47% water; (b) 0.3% sodium benzoate; and (c) 52% polyethylene glycol having an average molecular weight of 3350 Daltons.

5. The solution concentrate of claim 3, wherein the solution concentrate is packaged in a sealed container having a rate of permeation of oxygen into the container no greater than 0.1 cc/container/day from air at one atmosphere of pressure and 25° C., wherein the container is a bottle having a volumetric capacity of 1-50 ounces.

6. The solution concentrate of claim 5, wherein the container is a bottle having a volumetric capacity of 1-30 ounces, and the rate of permeation of oxygen into the bottle is no greater then 0.05 cc/container/day.

7. The solution concentrate of claim 5, wherein the container is a bottle having a volumetric capacity of 1-30 ounces, and the rate of permeation of oxygen into the bottle is no greater then 0.025 cc/container/day.

8. The solution concentrate of claim 3, wherein the pharmaceutically acceptable acid is a mineral acid selected from the group consisting of sulfuric acid, phosphoric acid, and hydrochloric acid.

9. The solution concentrate of claim 3, wherein at least 90% of the acid in the solution concentrate is hydrochloric acid.

10. The solution concentrate of claim 9, wherein the pharmaceutically acceptable acid is hydrochloric acid.

11. The solution concentrate of claim 3, wherein the solution concentrate contains one or more of the following degradation products in a detectable amount, each degradation product being present in an amount no greater than the following amount: (w/w): formic acid, 0.5%; ethylene glycol, 0.10%; diethylene glycol, 0.10%; formaldehyde, 200 ppm; and acetaldehyde, 200 ppm.

12. The solution concentrate of claim 3, wherein the solution concentrate contains one or more of the following degradation products in a detectable amount, each degradation product being present in an amount no greater than the following amount: (w/w): formic acid, 0.3%; ethylene glycol, 365 ppm; diethylene glycol, 590 ppm; formaldehyde, 175 ppm; and acetaldehyde, 115 ppm.

13. The solution concentrate of claim 3, wherein the solution concentrate contains one or more of the following degradation products in a detectable amount, each degradation product being present in an amount no greater than the following amount: (w/w): formic acid, 0.3%; ethylene glycol, 180 ppm; diethylene glycol, 180 ppm; formaldehyde, 70 ppm; and acetaldehyde, 70 ppm.

14. The solution concentrate of claim 13, wherein the amounts of the components and degradation products and the pH are within the specified ranges at the time when the solution concentrate is prepared and packaged.

15. The solution concentrate of claim 13, wherein the amounts of the components and degradation products and the pH are within the specified ranges after standing for 24 months at 25° C. and 40% relative humidity.

16. The solution concentrate of claim 13, wherein the amounts of the components and degradation products and the pH are within the specified ranges after standing for 24 months at 30° C. and 75% relative humidity.

17. The solution concentrate of claim 13, wherein the amounts of the components and degradation products and the pH are within the specified ranges after standing for 6 months at 40° C. and 20% relative humidity.

18. The solution concentrate of claim 3, wherein the solution concentrate further contains one or more pharmaceutically acceptable chelating agents.

19. The solution concentrate of claim 18, wherein the chelating agent is present in an amount between 0.01% w/w and 2.0% w/w.

20. The solution concentrate of claim 18, wherein the chelating agent is present in an amount between 0.01% w/w and 0.10% w/w.

21. The solution concentrate of claim 18, wherein the chelating agent is chosen from citric acid, malic acid or disodium edetate (EDTA) and mixtures thereof.

22. The solution concentrate of claim 21, wherein the chelating agent is disodium edetate (EDTA).

23. The solution concentrate of claim 12, wherein the pharmaceutically acceptable acid is hydrochloric acid.

24. The solution concentrate of claim 3, wherein the solution concentrate is packaged so that the oxygen concentration in the solution concentrate is not more than 5 ppm when the solution is first prepared and packaged.

25.-89. (canceled)
